(19) United States
(12) Patent Application Publication
(10) Pub. No.: US 2025/0361738 A1
(43) Pub. Date: Nov. 27, 2025

(54) HYBRID WATER MONITORING SYSTEM

(71) Applicant: ZODIAC POOL SYSTEMS LLC, Carlsbad, CA (US)

(72) Inventors: Anthony Levacque, Carlsbad, CA (US); Mark Bauckman, San Marcos, CA (US); Robert W. Stiles, Jr., Carlsbad, CA (US)

(21) Appl. No.: 19/216,694

(22) Filed: May 22, 2025

Related U.S. Application Data
(60) Provisional application No. 63/651,693, filed on May 24, 2024.

(51) Int. Cl. E04H 4/12; C02F 1/00; C02F 1/68

(52) U.S. Cl. CPC E04H 4/1209; C02F 1/008; C02F 1/685; C02F 2209/001

(57) ABSTRACT

A water quality monitoring system for a swimming pool or spa includes a first water sensing device and a second water sensing device. The first water sensing device may measure one or more water parameters via a probe reading, and the second water sensing device may measure one or more water parameters via a reagent reading.

### REFERENCE TO RELATED APPLICATION

This application claims the benefit of and priority to U.S. Provisional Patent Application No. 63/651,693, filed on May 24, 2024, and entitled HYBRID WATER MONITORING SYSTEM, the content of which is hereby incorporated by reference in its entirety.

### FIELD OF THE INVENTION

The invention relates to water testing or monitoring systems, and more particularly, but not necessarily exclusively, to water testing or monitoring systems for swimming pools or spas.

### BACKGROUND

Maintaining water quality is important for swimming pools, spas, hot tubs, and other water containing vessels (hereinafter “swimming pools or spas”) to avoid issues for users of the pool as well as equipment of the pool. For example, if the water chemistry of the swimming pool or spa is off, a health hazard may be posed to users and/or operation of various pool equipment and/or systems may be compromised.

Some conventional approaches to monitoring water parameters include reagent-based sensing using chemistry kits, remote testing, and maintenance service calls. Conventional chemistry kits may be complicated and not user-friendly to pool owners, and the individual using the chemistry kit may be uncertain of the results. Remote testing requires taking a sample of water to a store or chemistry laboratory to analyze the sample but may pose issues of sample contamination, improper storage, and/or a change in water chemistry during transit. In addition, reagent-based approaches are typically only performed periodically by a customer due to the volume of reagent required, but the periodic approach may lead to safety issues (e.g., the water may become very acidic or overchlorinated).

Another conventional approach includes the use of pH and oxidation reduction potential (ORP) probes. However, such probes have a limited lifetime and drift over time, and are often not a cost-effective way to measure all water parameters. In addition, such probes require significant maintenance for cleaning and calibration as they can be polluted (the junction, the glass bulb or tip of the sensors can be subject to chemical contamination, calcium deposit etc.), but customers commonly do not perform the required maintenance, which may lead to wrong measurements and/or safety issues (e.g., a very acidic pool or overchlorinated water, for example). In addition, it may be difficult to predict or troubleshoot an incorrect measurement from a probe, and the incorrect measurement may lead to over-dosing or under-dosing of chemicals into the pool.

As such, many existing approaches to monitoring water quality may be inconvenient, inaccurate, labor-intensive and time-consuming.

### SUMMARY

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various embodiments of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

According to certain embodiments, a water quality monitoring system includes a first water sensing device and a second water sensing device. The first water sensing device measures a water parameter via first measurement means, and the second water sensing device measures the water parameter via second measurement means different from the first measurement means.

According to some embodiments, a water quality monitoring system includes a first water sensing device configured to continuously measure a water parameter and a second water sensing device configured to periodically measure the water parameter.

According to various embodiments, a water quality monitoring system includes a first water sensing device configured to measure a water parameter via a probe reading and a second water sensing device configured to measure the water parameter via a reagent reading. In various embodiments, the water quality monitoring system may calibrate the first water sensing device based on the measurements from the second water sensing device.

According to certain embodiments, a water quality monitoring system includes a first water sensing device configured to measure a water parameter via a probe reading and a second water sensing device configured to measure the water parameter via a reagent reading. In certain embodiments, the water quality monitoring system may generate a control signal responsive to a difference between the measurement of the water parameter from the first water sensing device and the measurement of the water parameter from the second water sensing device.

According to some embodiments, a water quality monitoring system includes a first water sensing device configured to measure a first water parameter via a probe reading and a second water sensing device configured to measure a second water parameter via a reagent reading.

According to various embodiments, a method of monitoring a water parameter of a swimming pool or spa includes calibrating a first water sensing device configured to continuously measure a water parameter based on measurements from a second water sensing device configured to periodically measure the water parameter.

According to some embodiments, a method of monitoring a water parameter of a swimming pool or spa includes calibrating a first water sensing device configured to measure a water parameter via a probe reading based on measurements from a second water sensing device configured to measure the water parameter via a reagent reading.

According to various embodiments, a method of monitoring a water parameter of a swimming pool or spa includes generating an alert or control response based on a deviation of a measurement of a water parameter from a probe-based device compared to a measurement of the water parameter from a reagent-based device.

According to certain embodiments, a method of monitoring a water parameter of a swimming pool or spa includes providing a free chlorine measurement based on an ORP measurement from a probe-based device and based on pH and free chlorine measurements from a reagent-based device.

According to some embodiments, a method of monitoring a water parameter of a swimming pool or spa includes controlling a dosing system to control a first water parameter based on a measurement of the first water parameter by a probe and a measurement of a second water parameter by a reagent-based device.

Various implementations described in the present disclosure can include additional systems, methods, features, and advantages, which cannot necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIG. 1 illustrates a pool system according to embodiments.

FIG. 2 illustrates a method using the pool system of FIG. 1 according to embodiments.

FIG. 3 illustrates a method using the pool system of FIG. 1 according to embodiments.

FIG. 4 illustrates a method using the pool system of FIG. 1 according to embodiments.

FIG. 5 illustrates a method using the pool system of FIG. 1 according to embodiments.

FIG. 6 illustrates a method using the pool system of FIG. 1 according to embodiments.

### DESCRIPTION OF THE INVENTION

Described herein are systems and methods for monitoring water of a swimming pool or spa. In certain embodiments, the systems and methods described include a hybrid system utilizing two water sensing devices which are different types of water sensing devices. In some embodiments, a first water sensing device may provide a continuous reading of one or more water parameters, and a second water sensing device may provide a periodic and/or non-continuous reading of one or more water parameters. In some non-limiting examples, the first water sensing device may be a probe-based device and the second water sensing device may be a reagent-based device. In certain embodiments, the systems and methods described herein may calibrate the probe-based devices based on measurements of water parameters from the reagent-based device. In some embodiments, the systems and methods described herein may generate an alert and/or a control response based on a difference between a water parameter reading from the probe-based device and a water parameter reading from the reagent-based device.

The systems and methods described herein may provide accurate and reliable measurements of a plurality of water parameters including certain water parameters, such as but not limited to free chlorine, which traditionally are not performed by customers. In various embodiments, the systems and methods described herein may automatically control a dosing system and/or other suitable control system for controlling one or more water parameters based on the water parameter measurements from one or both sensing devices. Compared to traditional approaches, the systems and methods described herein may provide accurate and reliable measurements of water parameters, including water parameters that are not typically tested (such as but not limited to free chlorine). The systems and methods described herein may also provide accurate and reliable dosing and water parameter control compared to traditional approaches. The systems and methods described herein may also help identify when a water sensing device, such as a probe-based device, needs to be recalibrated or replaced. Various other benefits and advantages may be realized with the systems, devices, and methods provided herein, and the aforementioned advantages should not be considered limiting.

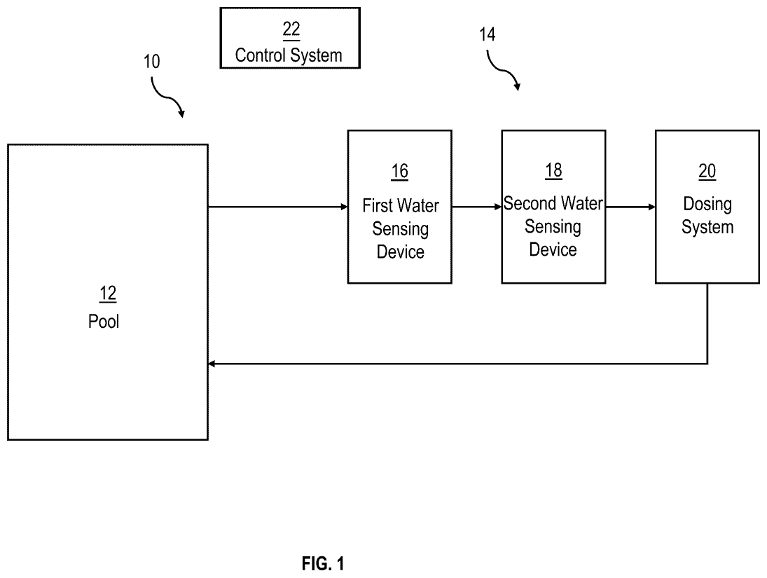

FIG. 1 illustrates an example of a pool system 10 according to embodiments. The pool system 10 generally includes a swimming pool or spa 12 (hereinafter “pool 12”) and a monitoring system 14 for measuring one or more water parameters of water of the pool 12. As non-limiting examples and as discussed in detail below, the monitoring system 14 may measure water parameters such as but not limited to pH, ORP, free chlorine, total chlorine, total alkalinity, and/or cyanuric acid concentration. In some embodiments, the monitoring system 14 may measure one water parameter, although in other embodiments, the monitoring system 14 may measure a plurality of water parameters, such as two water parameters, three water parameters, four water parameters, five water parameters, and/or more than five water parameters.

As illustrated in FIG. 1, the monitoring system 14 generally includes one or more first water sensing systems or devices 16 (hereinafter “first water sensing device 16”) and one or more second water sensing systems or devices 18 (hereinafter “second water sensing device 18”). Optionally, the monitoring system 14 includes a control or dosing system 20 for controlling one or more water parameters of the water of the pool 12. The number of first water sensing devices 16, second water sensing devices 18, and dosing systems 20 should not be considered limiting. Similarly, the location and/or arrangement of the first water sensing device 16, the second water sensing device 18, and the dosing system 20 should not be considered limiting. Moreover, while illustrated as separate devices in FIG. 1, in other embodiments, the monitoring system 14 may include a single device for water sensing and dosing and/or various combinations of devices as desired. As a non-limiting example, a single device may be configured and/or include features such that it is the first water sensing device 16, the second water sensing device 18, and the dosing system 20. As a further non-limiting example, the first and second water sensing devices 16, 18 may be provided as a single device, and the dosing system 20 may be provided as a separate device. As yet another non-limiting example, the first water sensing device 16 may be provided as a separate device, and a single device may include both the second water sensing device 18 and the dosing system 20. As such, reference to a first water sensing device and a second water sensing device does not require physically separate devices and instead may refer to a single device utilizing two (or more) water sensing technologies. Other combinations or sub-combinations may be implemented as desired.

In certain embodiments, the first water sensing device 16 and the second water sensing device 18 are different types of water sensing devices and/or are configured to measure water parameters at different measurement rates. In certain embodiments, the first water sensing device 16 and the second water sensing device 18 are each arranged to measure a same type and/or a same number of water parameters. As non-limiting examples, the first water sensing device 16 and the second water sensing device 18 each may measure one or more of pH, ORP, free chlorine, total chlorine, total alkalinity, and/or cyanuric acid concentration. Alternatively, the first water sensing device 16 and the second water sensing device 18 need not measure the same type of water parameter and/or the same number of water parameters. As non-limiting examples, the first water sensing device 16 may measure pH and ORP, and the second water sensing device 18 may measure free chlorine, pH, cyanuric acid, and total alkalinity. Other combinations of water parameters measured by the first and second water sensing devices 16, 18 may be utilized as desired.

In some embodiments, the first water sensing device 16 is a probe-based water sensing device and the second water sensing device 18 is a reagent-based water sensing device. In such embodiments, the first water sensing device 16 may include a sensor or probe in contact with water of the pool system 10 for measuring one or more water parameters. The second water sensing device 18 may be various devices or systems which utilize liquid reagents to measure one or more water parameters. In such embodiments, various reagent-based devices may be utilized.

Additionally, or alternatively, the first water sensing device 16 may perform measurements of one or more water parameters at a first measurement rate, and the second water sensing device 18 may perform measurements at a second measurement rate. As a non-limiting example, the first water sensing device 16 may continuously measure one or more water parameters, and the second water sensing device 18 may perform periodic and/or non-continuous measurements of one or more water parameters. In such embodiments with periodic measurements, the measurements may be performed at regular intervals, irregular intervals, and/or on demand as desired. In some embodiments, the first water sensing device 16 as a probe-based sensing device may be controlled to continuously measure one or more water parameters and the second water sensing device 18 as a reagent-based sensing device may be controlled to periodically measure one or more water parameters.

The dosing system 20 may be various devices or mechanisms suitable for controlling/adjusting one or more water parameters of water of the pool 12. As non-limiting examples, the dosing system 20 may dispense chemicals such as chlorine, acid (to lower pH), combinations thereof, and/or other chemicals and/or additives as desired into water of the pool system 10.

In some embodiments, and as illustrated in FIG. 1, the pool system 10 includes the control system 22 (one or more processing units and/or memory devices) communicatively coupled to one or more pieces of equipment of the monitoring system 14 (e.g., the first water sensing device 16, the second water sensing device 18, and/or the dosing system 20) using various communication techniques as desired. In some embodiments, in addition to the monitoring system 14, the control system 22 optionally may be communicatively coupled to other equipment and/or systems of the pool system 10 as desired. In certain embodiments, the control system 22 is communicatively coupled to at least one first water sensing device 16, at least one second water sensing device 18, and/or the dosing system 20. While the control system 22 is illustrated as local to the pool system 10 in FIG. 1, in other embodiments, the control system 22 may be remote from the pool system 10 (e.g., the control system 22 may be internet-based, in the cloud, etc.). Similarly, while the control system 22 is illustrated as a standalone device in FIG. 1, in other embodiments, one or more components of the control system 22 may be implemented in other components of the pool system 10 and/or remote from the pool system as desired. As a non-limiting example, the control system 22 optionally may be implemented locally onboard the first water sensing device 16, the second water sensing device 18, and/or the dosing system 20.

The one or more processing units of the control system 22 may be any suitable processing device or combinations of devices including but not limited to one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units, and/or a combination thereof. The one or more memory devices of the control system 22 may be any machine-readable medium that can be accessed by the processor, including but not limited to any type of long term, short term, volatile, nonvolatile, or other storage medium, and is not to be limited to any particular type of memory or number of memories, or type of media upon which memory is stored. Moreover, as disclosed herein, the term “storage medium,” “storage” or “memory” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to, portable or fixed storage devices, optical storage devices, wireless channels, and/or various other storage mediums capable of storing that contain or carry instruction(s) and/or data.

In certain embodiments, the control system 22 optionally includes an associated user interface, including but not limited to a human machine user interface, such that the control system 22 may obtain information from a user and/or provide information to the user via the user interface. When included, the user interface may be on the control system 22 itself or may be at a location remote from the control system 22 such as, but not limited to, another location within the pool system 10. Additionally, or alternatively, the control system 22 optionally may include one or more communication modules such that the control system 22 may receive and/or send information to a user device and/or other location. Non-limiting examples of communication modules may include systems and mechanisms enabling wired communication and/or wireless communication (e.g., near field, cellular, Wi-Fi, Bluetooth®, Bluetooth Low Energy (BLE), low-power wide area network, cloud-based communication etc.). In one non-limiting example, the control system 22 may receive and/or send information to a user device via an application running on the user device.

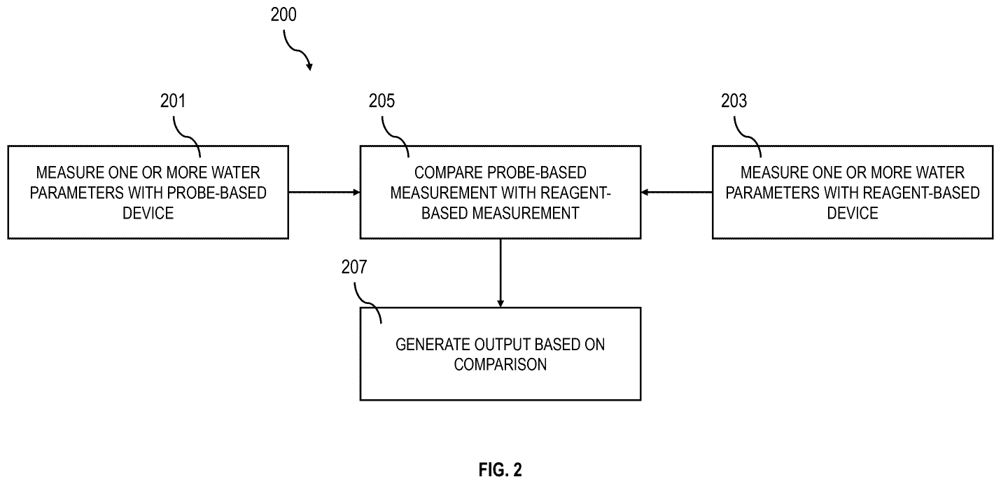

FIG. 2 illustrates an example of a monitoring process 200 using the monitoring system 14 according to various embodiments. The process is provided for illustrative purposes, and various other monitoring processes may be realized with the monitoring system 14 described herein.

In a block 201, the process 200 includes measuring one or more water parameters with the first water sensing device 16. In one non-limiting example, block 201 includes measuring pH and/or ORP of water from the pool system 10; however, in other embodiments, other water parameters and/or combinations of water parameters may be measured by the first water sensing device 16 as desired. In some embodiments, block 201 includes measuring the one or more water parameters with the probe-based sensing device 16, and the water parameters measured in block 201 are probe-based measurements. In certain embodiments, block 201 includes measuring the one or more water parameters with the first water sensing device 16 at a first measurement rate. In one non-limiting example, the first measurement rate may be a continuous measurement rate, and the one or more water parameters are continuously measured by the first water sensing device 16. In a non-limiting example, block 201 includes continuously measuring at least pH and/or ORP using a probe-based sensing device 16.

In a block 203, which may be performed concurrently with block 201, the process 200 includes measuring one or more water parameters with the second water sensing device 18. In one non-limiting example, block 203 includes measuring free chlorine, total chlorine pH, alkalinity, and/or cyanuric acid; however, in other embodiments, other water parameters and/or combinations of water parameters may be measured by the second water sensing device 18 as desired. In some embodiments, block 203 includes measuring the one or more water parameters with the reagent-based sensing device 18, and the water parameters measured in block 201 are probe-based measurements. In one non-limiting example, block 203 includes measuring the one or more water parameters using a reagent-based device such as a reagent testing system and/or a photometer. In some embodiments, block 203 includes measuring the one or more water parameters with the second water sensing device 18 at a second measurement rate that is different form the first measurement rate. In one non-limiting example, the second measurement rate may be a periodic and/or non-continuous measurement rate, and the one or more water parameters are periodically and/or non-continuously measured by the second water sensing device 18 compared to the continuous measurements of the first water sensing device 16. In one non-limiting example, the second measurement rate may be a daily rate, or one measurement of a water parameter per day. However, in other embodiments, other periodic measurement rates may be implemented as desired. In a non-limiting example, block 203 includes periodically measuring at least pH, alkalinity, cyanuric acid, and free chlorine using a reagent-based sensing device 18.

In a block 205, the water measurements from blocks 201 and 203 may be obtained (e.g., by the control system 22), and the process 200 includes comparing the water parameter measurements from the first water sensing device 16 to the water parameter measurements from the second water sensing device 18.

In a block 207, based on the comparison in block 205, the process 200 includes generating one or more outputs.

In some embodiments, block 207 includes maintaining a status quo responsive to the water parameter measurements from the first water sensing device 16 being within a threshold or acceptable deviation of the water parameter measurements from the second water sensing device 18. As a non-limiting example, block 207 may include maintaining a current state of the monitoring system 14 when a pH measurement from the first water sensing device 16 is within a threshold (or matches) a pH measurement from the second water sensing device 18.

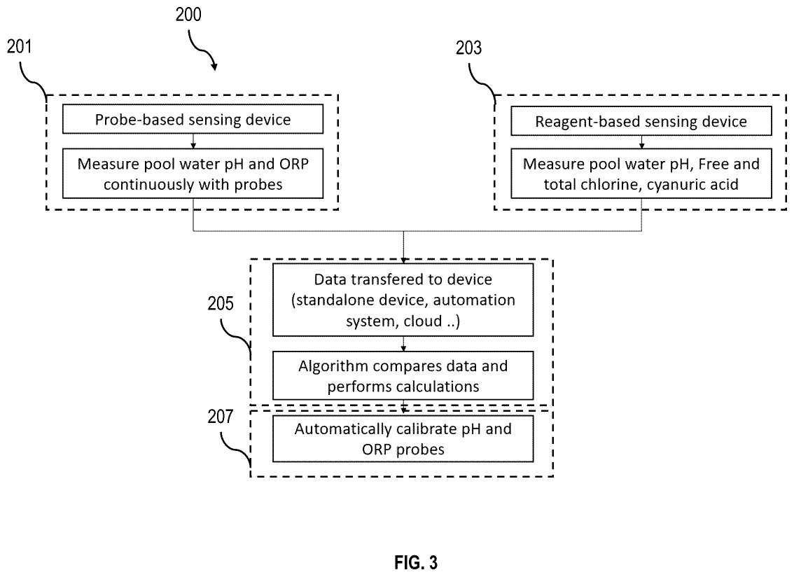

In various embodiments, block 207 includes calibrating the first water sensing device 16 based on the water parameter measurements from the second water sensing device 18. As a non-limiting example, pH and/or ORP measurements from the first water sensing device 16 may be compared to free chlorine, total chlorine, pH, and/or cyanuric acid measurements from the second water sensing device 18. In this example, the first water sensing device 16 measuring pH and/or ORP may be calibrated (e.g., to improve accuracy of the pH and/or ORP measurements) based on any deviations and/or calculations from the second water sensing device 18. FIG. 3 illustrates a specific implementation of the process in which the block 207 includes calibrating the first water sensing device 16.

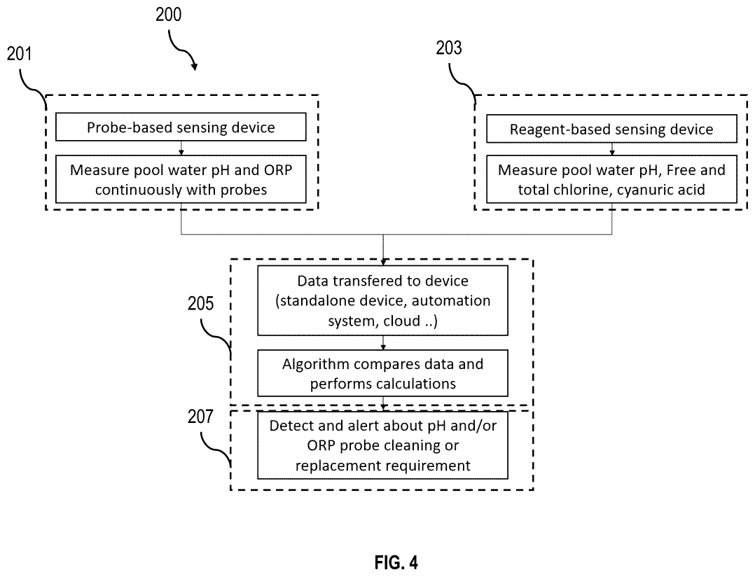

Additionally, or alternatively, block 207 includes generating an alert and/or implementing a maintenance process based on the comparison of the water parameter measurements from the first water sensing device 16 and the water parameter measurements from the second water sensing device 18. In some embodiments, generating the alert and/or implementing the maintenance process may be implemented after calibrating the first water sensing device 16 and responsive to a water parameter measurement from the calibrated first water sensing device 16 deviating and/or being outside of a threshold from the water parameter measurement from the second water sensing device 16. As a non-limiting example, the first water sensing device 16 may provide a pH and/or ORP measurement, which is compared to a corresponding measurement from the second water sensing device 18. In this example, the first water sensing device 18 may be calibrated based on a deviation from the measurement from the second water sensing device 18. In this example, a measurement from the calibrated first water sensing device 16 which is still deviating and/or outside of a threshold compared to the measurement from the second water sensing device may be identified as a maintenance event for the first water sensing device 16 (e.g., the first water sensing device 16 needs to be cleaned or replaced). An alert may be provided to the consumer via a user interface, a remote device, and/or as otherwise desired. Additionally, or alternatively, a maintenance process may be initiated automatically by the system. FIG. 4 illustrates a specific implementation of the process 200 in which the block 207 includes generating an alert and/or implementing a maintenance process for the first water sensing device 16.

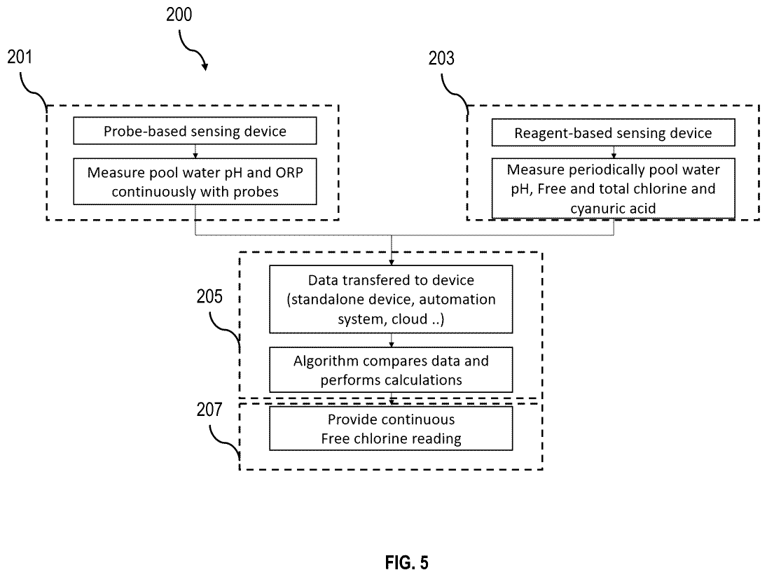

Additionally, or alternatively, block 207 includes generating an alert or notification with a water parameter measurement based on the comparison of the water parameter measurements from the first water sensing device 16 and the water parameter measurements from the second water sensing device 18. As a non-limiting example, block 207 may include providing a free chlorine measurement to a user (e.g., on a user interface, remote device, etc.) based on an ORP measurement from the first water sensing device 16 and a measurement of free chlorine, pH, and cyanuric acid from the second water sensing device 18. In certain embodiments, block 207 includes generating a correlation between free chlorine concentration and the ORP measurement to the consumer based on the comparison of the reagent-based measurements and the probe-based measurements. FIG. 5 illustrates a specific implementation of the process 200 in which the block 207 includes generating an alert or notification with a water parameter measurement.

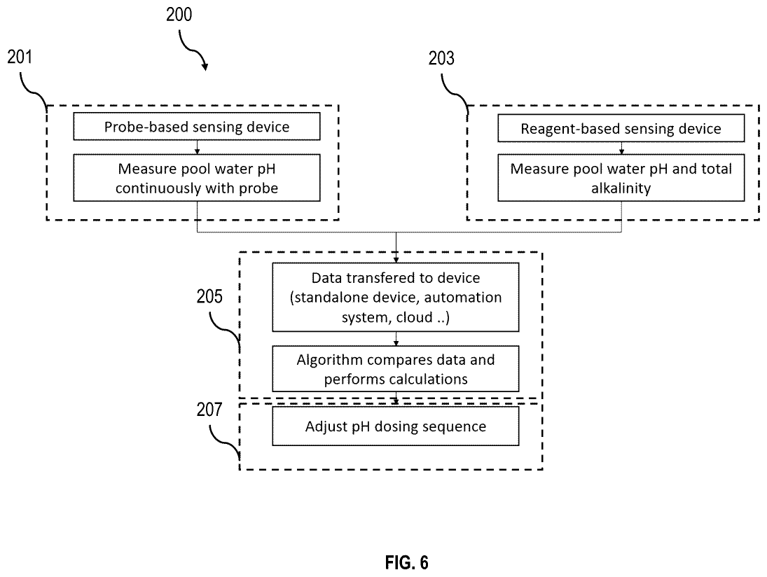

Additionally, or alternatively, block 207 includes controlling dosing by the dosing system 20 based on the comparison of the reagent-based measurements and the probe-based measurements. As a non-limiting example, a dosage of acid (or other chemical) from the dosing system into water of the pool 12 may be automatically controlled or adjusted based on a pH reading from the first water sensing device 16 and an alkalinity measurement from the second water sensing device 18. FIG. 6 illustrates a specific implementation of the process 200 in which the block 207 includes controlling the dosing from the dosing system 20.

Various other control responses or outputs may be realized in block 207, and the aforementioned examples should not be considered limiting.

The water monitoring system 14 with the first and second water sensing devices 16, 18 may provide various advantages and benefits compared to traditional approaches. In some embodiments, the water monitoring system 14 with the first and second water sensing devices 16, 18 may allow for continuous measurement of water parameters (e.g., using the first water sensing device 16) while maintaining an accuracy and reliability of such measurements. As non-limiting examples, the second water sensing device 18, which may make periodic measurements but with more accuracy, may be utilized to calibrate the first water sensing device 16. In some embodiments, such calibration may be performed at regular intervals, at irregular intervals, based on a performance of a measurement by the second water sensing device 18, and/or as otherwise desired.

Additionally, or alternatively, the water monitoring system 14 with the first and second water sensing devices 16, 18 may allow for improved identification and monitoring of the performance of the first water sensing device 16. As non-limiting examples, the water parameter measurements from the first water sensing device 16 may be compared to the water parameter measurements from the second water sensing device 18. In such embodiments, an alert and/or other control response may be generated based on a difference or deviation (e.g., relative to a threshold) of the water parameter measurements from the first water sensing device 16 compared to the water parameter measurements from the second water sensing device 18.

Additionally, or alternatively, the water monitoring system 14 with the first and second water sensing devices 16, 18 may allow for improved measurement and/or correlation of water parameters. As a non-limiting example, the water monitoring system 14 may provide a measurement of free chlorine based on an ORP measurement from the first water sensing device 16 and a measurement of free chlorine, pH, and cyanuric acid from the second water sensing device 18.

Additionally, or alternatively, the water monitoring system 14 with the first and second water sensing devices 16, 18 may allow for improved chemical dosing from the dosing system 20. As non-limiting examples, the dose of chemical from the dosing system 20 may be automatically adjusted based on the water parameter readings from the first water sensing device 16 and/or the second water sensing device 18. In some non-limiting examples, the dose from the dosing system 20 may be controlled based on a measurement of a first water parameter from the first water sensing device 16 and a measurement of a second water parameter from the second water sensing device 18.

Various other benefits and advantages may be realized with the systems, devices, and methods provided herein, and the aforementioned advantages should not be considered limiting.

Exemplary concepts or combinations of features of the invention may include:
A. A water quality monitoring system comprising a first water sensing system or device and a second water sensing system or device, wherein the first water sensing system or device measures a water parameter via first measurement means, and wherein the second water sensing system or device measures the water parameter via second measurement means different from the first measurement means.
B. A water quality monitoring system comprising:
   i. a first water sensing system or device configured to continuously measure a water parameter; and
   ii. a second water sensing system or device configured to periodically measure the water parameter.
C. A water quality monitoring system comprising:
   i. a first water sensing system or device configured to measure a water parameter via a probe reading; and
   ii. a second water sensing system or device configured to measure the water parameter via a reagent reading, wherein the water quality monitoring system is configured to calibrate the first water sensing system or device based on the measurements from the second water sensing system or device.
D. A water quality monitoring system comprising:
   i. a first water sensing system or device configured to measure a water parameter via a probe reading; and
   ii. a second water sensing system or device configured to measure the water parameter via a reagent reading, wherein the water quality monitoring system is configured to generate a control signal responsive to a difference between the measurement of the water parameter from the first water sensing system or device and the measurement of the water parameter from the second water sensing system or device.
E. A water quality monitoring system comprising:
   i. a first water sensing system or device configured to measure a first water parameter via a probe reading; and
   ii. a second water sensing system or device configured to measure a second water parameter via a reagent reading.
F. The water quality monitoring system of any preceding or subsequent statement or combination of statements, wherein the first water sensing system or device and the second water sensing system or device are physically separate devices.
G. The water quality monitoring system of any preceding or subsequent statement or combination of statements, wherein the first water sensing system or device and the second water sensing system or device are provided as components of a single device.
H. The water quality monitoring system of any preceding or subsequent statement or combination of statements, wherein the first water sensing system or device is a probe and the second water sensing system or device is a photometer.
I. The water quality monitoring system of any preceding or subsequent statement or combination of statements, wherein the water parameters include but are not limited to pH, free chlorine, total chlorine, total alkalinity, and/or cyanuric acid concentration.
J. The water quality monitoring system of any preceding or subsequent statement or combination of statements, further comprising a dosing system for controlling one or more water parameters, wherein the dosing system is controlled based on water parameters measured by the first water sensing device.
K. The water quality monitoring system of any preceding or subsequent statement or combination of statements, wherein the first water sensing system and the second water sensing system are components of a common device.
L. The water quality monitoring system of any preceding or subsequent statement or combination of statements, wherein the first water sensing system is a first water sensing device and the second water sensing system is a second water sensing device different from the first water sensing device.
M. A method of monitoring a water parameter of a swimming pool or spa, the method comprising calibrating a first water sensing system or device configured to continuously measure a water parameter based on measurements from a second water sensing system or device configured to periodically measure the water parameter.
N. A method of monitoring a water parameter of a swimming pool or spa, the method comprising calibrating a first water sensing system or device configured to measure a water parameter via a probe reading based on measurements from a second water sensing system or device configured to measure the water parameter via a reagent reading.
O. A method of monitoring a water parameter of a swimming pool or spa, the method comprising generating an alert or control response based on a deviation of a measurement of a water parameter from a probe-based system or device compared to a measurement of the water parameter from a reagent-based system or device.
P. A method of monitoring a water parameter of a swimming pool or spa, the method comprising providing a free chlorine measurement based on an ORP measurement from a probe-based system or device and based on pH and free chlorine measurements from a reagent-based system or device.
Q. The method of any preceding or subsequent statement or combination of statements, wherein the free chlorine measurement is optionally based on a cyanuric acid measurement from the reagent-based system or device.
R. A method of monitoring a water parameter of a swimming pool or spa, the method comprising controlling a dosing system to control a first water parameter based on a measurement of the first water parameter by a probe and a measurement of a second water parameter by a reagent-based system or device.
S. The method of any preceding or subsequent statement or combination of statements, wherein the first water parameter comprises pH, and wherein the second water parameter comprises alkalinity.

These examples are not intended to be mutually exclusive, exhaustive, or restrictive in any way, and the invention is not limited to these example embodiments but rather encompasses all possible modifications and variations within the scope of any claims ultimately drafted and issued in connection with the invention (and their equivalents). For avoidance of doubt, any combination of features not physically impossible or expressly identified as non-combinable herein may be within the scope of the invention. Further, although applicant has described devices and techniques for use principally with swimming pools or spas, persons skilled in the relevant field will recognize that the present invention conceivably could be employed in connection with other water containing vessels and in other manners, particularly but not limited to underwater installations. Finally, references to “pools” and “swimming pools” herein may also refer to spas or other water containing vessels used for recreation, training, or therapy.

That which is claimed:

1. A water quality monitoring system comprising a first water sensing system and a second water sensing system, wherein the first water sensing system measures a water parameter via first measurement means, and wherein the second water sensing system measures the water parameter via second measurement means different from the first measurement means.

2. The water quality monitoring system of claim 1, wherein the first measurement means comprises a probe reading, and wherein the second measurement means comprises a reagent-based reading.

3. The water quality monitoring system of claim 2, wherein the water quality monitoring system is configured to calibrate the first water sensing system based on measurements from the second water sensing system.

4. The water quality monitoring system of claim 1, wherein the first water sensing system is configured to continuously measure the water parameter and the second water sensing system is configured to periodically measure the water parameter.

5. The water quality monitoring system of claim 1, wherein the water quality monitoring system is configured to generate a control signal responsive to a difference between the measurement of the water parameter from the first water sensing system and the measurement of the water parameter from the second water sensing system.

6. The water quality monitoring system of claim 1, wherein the water parameter comprises one or more of pH, free chlorine, total chlorine, total alkalinity, and/or cyanuric acid concentration.

7. The water quality monitoring system of claim 1, wherein the first water sensing system and the second water sensing system are components of a common device.

8. The water quality monitoring system of claim 1, wherein the first water sensing system is a first water sensing device and the second water sensing system is a second water sensing device different from the first water sensing device.

9. A water quality monitoring system comprising:
a first water sensing system configured to continuously measure a water parameter; and
a second water sensing system configured to periodically measure the water parameter.

10. The water quality monitoring system of claim 9, wherein the water quality monitoring system is configured to calibrate the first water sensing system based on the measurements from the second water sensing system.

11. The water quality monitoring system of claim 9, wherein the water quality monitoring system is configured to generate a control signal responsive to a difference between the measurement of the water parameter from the first water sensing system and the measurement of the water parameter from the second water sensing system.

12. The water quality monitoring system of claim 9, wherein the first water sensing system is a probe and the second water sensing system is a photometer.

13. The water quality monitoring system of claim 9, wherein the water parameters include one or more of pH, free chlorine, total chlorine, total alkalinity, and/or cyanuric acid concentration.

14. The water quality monitoring system of claim 9, further comprising a dosing system for controlling one or more water parameters, wherein the dosing system is controlled based on water parameters measured by the first water sensing device.

15. The water quality monitoring system of claim 9, wherein the first water sensing system is a first water sensing device and the second water sensing system is a second water sensing device different from the first water sensing device.

16. A water quality monitoring system comprising:
a first water sensing system configured to measure a first water parameter via a probe reading; and
a second water sensing system configured to measure a second water parameter via a reagent reading.

17. The water quality monitoring system of claim 16, wherein the first and second water parameters are selected from pH, free chlorine, total chlorine, total alkalinity, and/or cyanuric acid concentration.

18. The water quality monitoring system of claim 16, further comprising a dosing system for controlling one or more water parameters, wherein the dosing system is controlled based on water parameters measured by the first water sensing device.

19. The water quality monitoring system of claim 16, wherein the first water sensing system is configured to continuously measure the first water parameter and the second water sensing system is configured to periodically measure the second water parameter.

20. The water quality monitoring system of claim 16, further comprising a dosing system, wherein the water quality monitoring system is configured to control the first water parameter based on the measurement of the first water parameter by the first water sending device and the measurement of the second water parameter by the second water sensing device.

21. The water quality monitoring system of claim 16, wherein the first water sensing system is a first water sensing device and the second water sensing system is a second water sensing device different from the first water sensing device.
